(19) United States
(12) Patent Application Publication
(10) Pub. No.: US 2008/0195113 A1
(43) Pub. Date: Aug. 14, 2008

(54) Bone Cement Delivery Device

(75) Inventor: George Sikora, Bridgewater, MA (US)

Correspondence Address: GROSSMAN, TUCKER, PERREAULT & PFLEGER, PLLC, 55 SOUTH COMMERICAL STREET, MANCHESTER, NH, 03101, US

(73) Assignee: ARTHROSURFACE INCORPORATED, Franklin, MA (US)

(21) Appl. No.: 12/031,534

(22) Filed: Feb. 14, 2008

Related U.S. Application Data
(60) Provisional application No. 60/889,927, filed on Feb. 14, 2007.

(51) Int. Cl. A61B 17/58

(52) U.S. Cl. 606/93

(57) ABSTRACT

A device and method for dispensing an adhesive wherein a housing includes a proximal portion and distal portion, an opening is defined in the distal portion of the housing, and a housing lumen is defined within the housing and extends through the opening. The device may also include a syringe including a body defining a syringe lumen and a delivery tip, wherein at least a portion of the body is positionable within the housing lumen. Furthermore, a screw including a proximal end and a distal end and a plunger are positionable within the syringe lumen.

### CROSS-REFERENCE TO RELATED APPLICATIONS

The present application claims the benefit of the filing date of U.S. Provisional Patent Application No. 60/889,927 filed on Feb. 14, 2007, the teachings of which are incorporated herein by reference.

### FIELD

The present disclosure is directed to a bone cement delivery device, and, more particularly, to a mechanically assisted bone cement delivery device.

### BACKGROUND

As is well understood in the orthopedic arts, bone cement is frequently used to affix artificial implant devices directly to bone. Typically, bone cement is a highly viscous material that, not only is difficult to work with, but is generally difficult to deliver precisely, especially in tight working areas.

### BRIEF DESCRIPTION OF DRAWINGS

Features and advantages of the claimed subject matter will be apparent from the following detailed description of embodiments consistent therewith, which description should be considered with reference to the accompanying drawings, wherein:

FIG. 1 is an exploded view of a bone cement delivery device according to one embodiment; and

FIG. 2 is an exploded view of another bone cement delivery device according to another embodiment.

Although the following Detailed Description will proceed with reference being made to illustrative embodiments, many alternatives, modifications, and variations thereof will be apparent to those skilled in the art.

### DETAILED DESCRIPTION

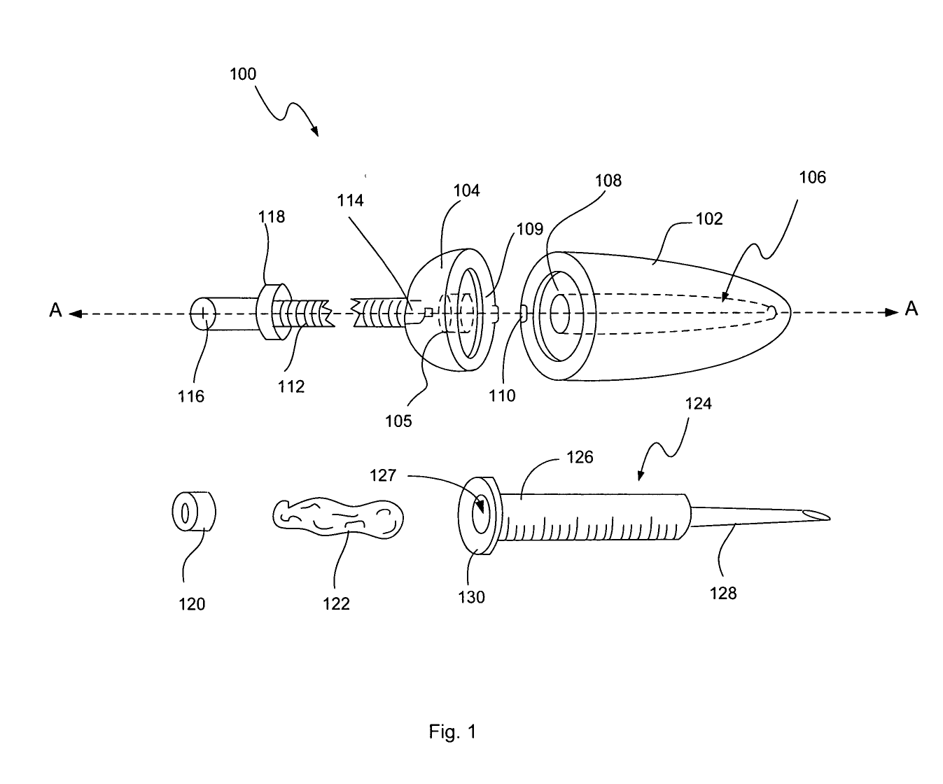

FIG. 1 depicts an exploded view of a bone cement delivery device 100 consistent with one embodiment of the present disclosure. Generally, device 100 may include a syringe housing 102 and a locking end cap 104. Syringe housing 102 may define a lumen 106 dimensioned to receive a syringe therein. The lumen 106 may taper at the distal end thereof and extend through an opening defined in the distal end of the housing 102. A bore 108 disposed on one face of the housing 102 may be configured to mate with an ear portion of a syringe, as will be described below, to prevent rotation of the syringe within the housing. Housing 102 may also include a locking feature 110 configured to mate with a locking feature 109 of the end cap 104, as will be described below.

Locking end cap 104 may include a screw member 112 that may be advanced through an orifice in the proximal end of the cap 104. The orifice may include a threaded portion 105 to receive the screw threads. The threaded screw portion may be removable or exhibit a higher hardness than the remainder of the locking cap. Locking end cap 104 may also include a locking feature 109 configured to mate with locking feature 110 of housing 102. Locking features 109/110 may include, for example, a J-latch type lock and/or latch, and/or other locking feature to permit end cap 104 to be removably coupled to housing 102. The distal end of the screw 112 may include an abutment 114 that is mated with a plunger 120. The screw 112 may be mated in either a non-rotating or rotating fashion with the plunger 120, such that as the screw 112 turns, the plunger 120 may either rotate with the screw 112 or remain stationary. The screw 112 may also include a stop collar 118, positioned at a predetermined point along the length of the screw, that prevents the screw 112 from being advanced into housing 102 beyond a predetermined distance (i.e., stop collar prevents further advancement of the screw 112 once the stop collar abuts the end cap 104). The proximal end of the screw 112 may include a head 116 that may be engaged by a driving device, for example, an electric screwdriver (not shown), hand screwdriver (not shown), etc. To that end, head 116 may include any variety of male and/or female engagement features, for example, Philips, flat, Allen, Torx, Hex and/or other engagement feature configured to mechanically engage a driving device. In addition, the engagement feature may include a lever for hand rotation of the screw, such as one or more elongate projections extending from the head of the screw substantially perpendicular to the axis of screw rotation, axis A-A.

It should be noted that overall shape of the housing 102 and end cap 104 may be designed to permit a human hand to hold the housing firmly to prevent rotation of the housing when operably coupled to a driving device. To that end, although not shown in the figures, housing 102 may include grip material or mating features that mate with portions of a human hand to enhance delivery control of bone cement. It may be appreciated that the entire housing may be made of grip material or specific portions of the housing may be made of grip material. The grip material may be formed of an elastomeric material including silicone, i.e., siloxane based, polymers, thermoplastic elastomers, urethanes, natural or synthetic rubber, etc. The mating features may include a number of projections and/or depressions in the outer surface of the housing. In one example, the projections and/or depressions may accommodate the geometry of a hand, be it a left or right hand, including the fingers and/or palm.

The delivery device 100 of this embodiment may also include a syringe 124 that is dimensioned to be disposed within syringe housing 102. The syringe 124 may be any type of syringe known in the art, and may generally include a body portion 126 defining a lumen 127, a delivery tip 128 and an ear portion 130. As alluded to above, ear portion 130 may have a shape that mates with bore 108, for example a D-shape that mates with bore 108. Such shape may prevent the syringe from twisting in the housing during delivery and retains the syringe 124 in a non-rotatable manner relative to the housing 102. Bone cement 122 may be disposed within lumen 127. Plunger 120 may be dimensioned to fit snugly into lumen 127 to push bone cement 122 through the syringe.

In operation, an appropriate/desired amount of bone cement 122 may be placed into the syringe 124 and plunger 120 may be placed over the bone cement and within the lumen 127. The syringe 126 may be placed within the lumen 106 of housing 102 and disposed so that the delivery tip 128 extends from the distal opening of the housing 102. Ear 130 and bore 108 may mate to prevent rotation of the syringe 126 within the housing 102. End cap 104 may be placed over the open face of the housing 102 and locked into place. To deliver bone cement, screw 112 may be engaged by a driving device to drive the screw 112 into the housing, push against plunger 120 and expel bone cement 122 from the delivery tip 128.

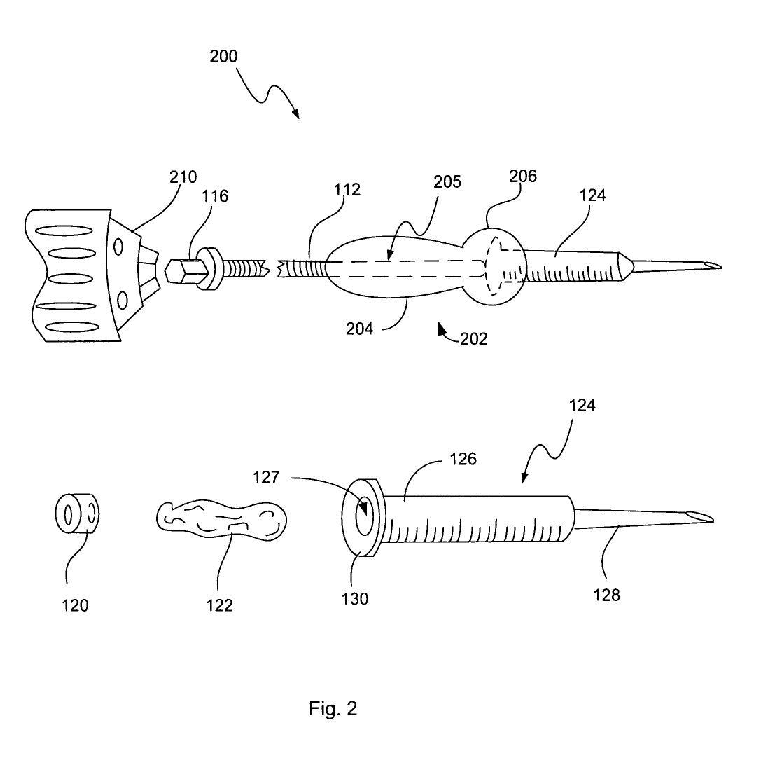

FIG. 2 depicts an exploded view of a bone cement delivery device 200 consistent with another embodiment of the present disclosure. This embodiment is similar to the embodiment of FIG. 1, and like components have been assigned like reference numerals. In this embodiment, housing 202 includes a handle portion 204 and a head portion 206. Handle portion 204 defines a lumen 205 configured to receive a syringe 124. The syringe 124 may be passed through the lumen 205 into the head portion 206 such that most of the syringe 124 extends from the distal end of the housing 202. The head portion 202 may include a mating feature therein (not shown) to mate with the ear 130 of the syringe 124 (similar to bore 108 of FIG. 1). Screw 112 may be advanced through the handle portion 204 to engage plunger 120 of the syringe 124 disposed partially within the head portion 206. Also shown in this embodiment is a chuck 210 of an electric drill configured to be coupled to the head 116 of screw 112.

It should be appreciated that while the above is in example directed to the application and dispensing of bone cement it should be appreciated that other adhesives, may be dispensed by the device, system and method disclosed herein. Furthermore, reference herein to an adhesive herein includes bone cement as well as other single or multiple component systems that may provide molecular, chemical and/or mechanical adhesion.

The adhesive may be any suitable material, such as adhesives based on vinyl type monomers, polymers and/or copolymers including acrylic and/or styrene based adhesives. The adhesives may exhibit a viscosity in the range of 1,000 cP to 100,000,000 cP at a temperature of at a temperature of 25° C. and a shear rate in the range of 0.1 s⁻¹ to 1,000 s⁻¹. Where the adhesive may be an acrylic based adhesive, and for example a poly(acrylic acid ester), such as poly(methacrylic acid ester) and/or poly(methyl methacrylate) adhesive, properties may be measured according to ASTM standard F451-99 or F451-99e1 (2007) or ISO standard 5833:1992 or 5833:2002.

Thus, in one aspect, the above delivery device may include a housing including a proximal portion and distal portion, an opening defined in the distal portion of the housing and a housing lumen defined within the housing and extending through the opening. In addition, the delivery device may include a syringe including a body defining a syringe lumen and a delivery tip, wherein a portion of the body may be positionable within the housing lumen. Furthermore, the delivery device may include a screw including a proximal end and a distal end positionable within the syringe lumen, and a plunger positionable within the syringe lumen. The delivery tip may be positioned outside of the housing.

In one example, the proximal portion of the housing may define a handle. In another example a bore may be defined in the housing, wherein the syringe may include an ear portion that mates with the bore. In a further example, the syringe may be retained in the housing in a non-rotate-able manner relative to the housing.

In another example, an end cap mate-able with the housing may be provided. The end cap may further include an orifice for receiving the screw. In addition, the housing may include a first locking feature and the end cap includes a second locking feature, wherein the first and second locking features are configured to mate.

In a further example, the screw may include a stop collar. In addition, the proximal end of the screw may include a head configured to be engaged with a driving device. Furthermore, the distal end of the screw may include an abutment to mate with the plunger. The plunger may be positionable within the syringe lumen in a non-rotating manner.

In another aspect, a method of dispensing an adhesive may include placing at least a portion of a syringe including a syringe lumen and a delivery tip in a housing, inserting a plunger into the syringe lumen, positioning a screw within the syringe lumen, advancing a screw in the syringe lumen, and advancing the plunger in the syringe lumen. The method may also include filling the syringe lumen with adhesive.

In one example, the syringe may be retained within the housing in a non-rotatable manner. The syringe may include an ear and the housing may include a bore to receive the ear and mating the ear in the bore. In addition, an end cap may be mated to the housing to retain the syringe within the housing.

In another example, the screw may include a stop collar, wherein the plunger maybe advanced until the stop collar abuts the housing. In addition, the screw may mate with the plunger. Furthermore, the plunger may be advanced in a non-rotating manner with respect to the syringe.

The foregoing description has been presented for purposes of illustration. It is not intended to be exhaustive or to limit the invention to the precise steps and/or forms disclosed, and obviously many modifications and variations are possible in light of the above teaching. It is intended that the scope of the invention be defined by the claims appended hereto.

What is claimed is:

1. A delivery device comprising:
a housing including a proximal portion and distal portion;
an opening defined in said distal portion of said housing;
a housing lumen defined within said housing and extending through said opening;
a syringe including a body defining a syringe lumen and a delivery tip, wherein a portion of said body is positionable within said housing lumen;
a screw including a proximal end and a distal end positionable within said syringe lumen, wherein said proximal end of said screw includes a head configured to be engaged with a driving device; and
a plunger positionable within said syringe lumen.

2. The delivery device of claim 1, wherein said proximal portion of said housing defines a handle.

3. The delivery device of claim 1, further comprising a bore defined in said housing, wherein said syringe includes an ear portion that mates with said bore.

4. The delivery device of claim 1, wherein said syringe is retained in said housing in a non-rotate-able manner relative to said housing.

5. The delivery device of claim 1, further comprising an end cap mate-able with said housing.

6. The delivery device of claim 5, wherein said end cap further comprises an orifice for receiving said screw.

7. The delivery device of claim 5, wherein said housing includes a first locking feature and said end cap includes a second locking feature, wherein said first and second locking features are configured to mate.

8. The delivery device of claim 1, wherein said screw includes a stop collar.

9. The delivery device of claim 1, wherein said driving device is selected from the group consisting of a screw driver or a drill.

10. The delivery device of claim 1, wherein said plunger is positionable within said syringe lumen in a non-rotating manner.

11. The delivery device of claim 1, wherein said distal end of said screw includes an abutment to mate with said plunger.

12. The delivery device of claim 1, wherein said delivery tip is positioned outside of said housing.

13. The delivery device of claim 1, wherein said syringe lumen includes an adhesive having a viscosity in the range of about 1,000 cP to 100,000,000 cP, wherein said viscosity is measured at a temperature of 25° C. and a shear rate of 0.1 s⁻¹ to 1,000 s⁻¹.

14. A method of dispensing an adhesive comprising:
placing at least a portion of a syringe including a syringe lumen and a delivery tip in a housing;
inserting a plunger into said syringe lumen;
positioning a screw within said syringe lumen;
advancing said screw in said syringe lumen via a driving device; and
advancing said plunger in said syringe lumen.

15. The method of claim 14, further comprising filling said syringe lumen with adhesive.

16. The method of claim 14, wherein said syringe includes an ear and said housing includes a bore to receive said ear and mating said ear in said bore.

17. The method of claim 14, wherein said screw includes a stop collar and said plunger is advanced until said stop collar abuts said housing.

18. The method of claim 14, further comprising retaining said syringe within said housing in a non-rotatable manner.

19. The method of claim 14, further comprising mating an end cap to the housing to retain said syringe within said housing.

20. The method of claim 14, wherein said screw mates with said plunger.

21. The method of claim 14, wherein said plunger is advanced in a non-rotating manner with respect to said syringe.

22. The method of claim 14, wherein said syringe lumen includes an adhesive having a viscosity in the range of about 1,000 cP to 100,000,000 cP, wherein said viscosity is measured at a temperature of 25° C. and a shear rate of 0.1 s⁻¹ to 1,000 s⁻¹.
